(19) United States
(12) Patent Application Publication
(10) Pub. No.: US 2007/0167616 A1
(43) Pub. Date: Jul. 19, 2007

(54) Salts of laked monoazo compounds

(76) Inventors: Ursula Luterbacher, Reinach (CH); Thomas Ruch, Delemont (CH); Christine Price, Riehen (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 11/587,690

(22) PCT Filed: Apr. 18, 2005
(86) PCT No.: PCT/EP05/51684

§ 371 Date: Oct. 26, 2006

(30) Foreign Application Priority Data
Apr. 27, 2004 (EP) 04101754.2

(51) Int. Cl. C07C 245/00

(52) U.S. Cl. 534/838

(57) ABSTRACT

The present invention relates to monoazo compounds of formula (1) wherein R₁ is hydrogen, halogen, especially chlorine, or C₁-C₄alkyl, especially methyl, and R₂ is halogen, especially chlorine, or C₁-C₄alkyl, especially methyl, preferably one of the substituents R₁ and R₂ being halogen and the other substituent being C₁-C₄alkyl, which are present in the form of salts or mono-, di-, tri- or tetra-valent mixed salts of different cations, such as, for example, Ca²⁺, Na⁺, NH₄⁺, NR₄⁺, H⁺, Li⁺, K⁺, Mg²⁺, Ba²⁺, Sr²⁺, Al³⁺, Pb²⁺, Mn²⁺, Zn²⁺, Cr²⁺, Co²⁺, Fe²⁺, Fe³⁺, Zr⁴⁺ and Cu²⁺ wherein R is C₁-C₆alkyl, in freely selected ratios, to processes for their preparation and to their use in the mass-pigmenting of organic materials and in the production of toners, wax transfer ribbons or colour filters.

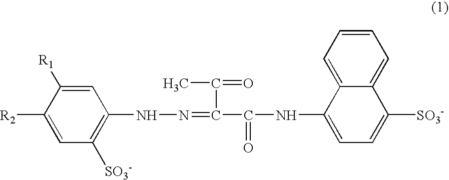

The present invention relates to novel monoazo compounds, to processes for their preparation and to their use as colorants, especially in the colouring of high molecular weight material.

A large number of monoazo compounds that can be used as pigments are known.

The increasingly high demands being made of the quality of colorations, for example the fastness properties, or the performance properties, for example the oversprayability, have resulted in there being a continuing need for new pigments having improved properties, especially in respect of fastness properties.

The aim of the present invention is therefore to provide novel, improved pigments based on monoazo compounds that can be used especially in the production of surface coatings, printing inks and colour filters or in the colouring of plastics. The novel pigments should yield colorations having high purity of shade, high colour strength and good fastness to overspraying.

It has been found that the desired aim is substantially achieved by the novel monoazo compounds defined hereinbelow.

The present invention accordingly relates to a monoazo compound of formula 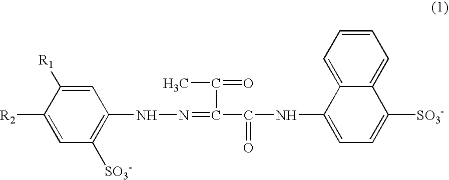
wherein
R₁ is hydrogen, halogen, especially chlorine, or C₁-C₄alkyl, especially methyl, and
R₂ is halogen, especially chlorine, or C₁-C₄alkyl, especially methyl,
preferably one of the substituents R₁ and R₂ being halogen and the other substituent being C₁-C₄alkyl,
which is present in the form of a salt or a mono-, di-, tri- or tetra-valent mixed salt of different cations, such as, for example, Ca²⁺, Na⁺, NH₄⁺, NR₄⁺, H⁺, Li⁺, K⁺, Mg²⁺, Ba²⁺, Sr²⁺, Al³⁺, Pb²⁺, Mn²⁺, Zn²⁺, Cr²⁺, Co²⁺, Fe²⁺, Fe³⁺, Zr⁴⁺ and Cu²⁺ wherein R is C₁-C₆alkyl, in a freely selected ratio, and has very good high-temperature stability in HDPE and a greenish-yellow shade.

Preferred as R₁ is halogen, especially chlorine, or C₁-C₄alkyl, especially methyl.

R as C₁-C₆alkyl is e.g. methyl, ethyl, n-propyl, isopropyl, n-butyl, isobutyl, sec-butyl, tert-butyl, pentyl or hexyl.

Preferred as R is methyl, ethyl, n-propyl, isopropyl, n-butyl, isobutyl, sec-butyl, tert-butyl, especially ethyl and more especially methyl.

The mixed salts according to the invention may be in the form of ternary salts, e.g. in the form of Ca++/NH₄⁺/Na⁺ or Ca⁺⁺/K⁺/Na⁺ mixed salts.

The mixed salts according to the invention preferably contain two different cations.

In a mixed salt containing two cations, the ratio of the cations may vary within a wide range, e.g. from 99.5 to 0.5 mol % of one cation to from 0.5 to 99.5 mol % of the other cation, preferably from 70 to 30 mol % of one cation to from 30 to 70 mol % of the other cation, more especially from 60 to 40 mol % of one cation to from 40 to 60 mol % of the other cation.

Preference is given to the monoazo compound of formula (1) present in the form of a Ca²⁺/Na⁺, Ca²⁺/NH₄⁺ or Ca²⁺/K⁺ mixed salt.

Of those mixed salts, preference is given to those having a Ca²⁺ content of >40 mol %, based on the total molar amount of the cations.

Of importance are Ca²⁺/NH₄⁺ mixed salts that have a NH₄+content of from 5 to 50 mol %, especially from 8 to 30 mol %, more especially from 10 to 30 mol %, based on the total molar amount of cations in the form of mixed crystals.

Also of importance are Ca²⁺/K⁺ mixed salts that have a K⁺ content of from 5 to 50 mol %, especially from 8 to 30 mol %, more especially from 10 to 30 mol %, based on the total molar amount of cations in the form of mixed crystals.

Likewise of importance are Ca²⁺/Na⁺ mixed salts that have a Na+content of from 5 to 60 mol %, especially from 10 to 55 mol %, based on the total molar amount of cations in the form of mixed crystals.

Also of importance is the monoazo compound of formula (1) which is in the form of the Ca²⁺ salt.

The present invention relates also to a process for the preparation of the monoazo compounds of formula (1) according to the invention.

They are prepared, for example, by diazotisation of 2-amino4-methyl-5-chlorobenzenesulfonic acid, 2-amino4-chloro-5-methylbenzenesulfonic acid or 2-amino-4,5-dichlorobenzenesulfonic acid or a salt or a mixed salt thereof, and coupling to 4-acetoacetylaminonaphtholsulfonic acid salt.

The monoazo compounds of formula (1) according to the invention exhibit substantially better fastness properties than similar known products and are therefore excellently suitable as tinctorially strong and high-temperature stable pigments.

The monoazo compounds of formula (1) according to the invention are distinguished especially by high fastness to migration, to water and to solvents, stability to light, to weathering and to high temperatures, good rheology and ready dispersibility, those advantageous properties being achievable, wholly unexpectedly and in contrast to products known hitherto, also in combination with high colour strength. The colour saturation (chroma C*) is also astonishingly high.

The monoazo compounds of formula (1) according to the invention exhibit outstanding crystallinity. Nevertheless, they may optionally be subjected to additional aftertreatment in order to optimise their properties still further. That step may be carried out according to processes known per se, for example by heating in water, a slightly polar hydrophilic organic solvent or a mixture thereof, to a temperature of approximately from 50 to 200° C. (optionally under pressure) for a period that may be from a few minutes up to 100 hours depending on the recrystallising medium and the temperature. Preference is given to after-treatment in water for from 0.5 to 6 hours at from 50 to 99° C., especially for from 1 to 4 hours at from 65 to 85° C. After-treatment is preferably carried out directly after laking, optionally without intermediate isolation.

The monoazo compounds of formula (1) according to the invention can be isolated in pure form and dried, following which they are readily dispersible in plastics, surface coatings and printing inks, for example by means of a ball mill or bead mill. They can also be used in the form of moist press cakes, without further processing, for the preparation of pigment dispersions. Conventional additives in conventional concentrations may be added to the monoazo compounds of formula (1) according to the invention, as required, before or during precipitation or isolation in order to improve the application-related properties.

As well as being suitable for the mass-pigmenting of high molecular weight organic materials in the form of plastics, surface coatings and printing inks, the monoazo compounds of formula (1) according to the invention are also suitable, for example, for the production of solid toners, wax transfer ribbons or colour filters.

The high molecular weight organic material to be coloured according to the invention may be of natural or synthetic origin and usually has a molecular weight in the range of from 10³ to 10⁸ g/mol. It may be, for example, a natural resin or drying oil, rubber or casein, or a modified natural material such as chlorinated rubber, an oil-modified alkyd resin, viscose, a cellulose ether or ester, such as cellulose acetate, cellulose propionate, cellulose acetobutyrate or nitrocellulose, but especially a totally synthetic organic polymer (both thermosetting plastics and thermoplastics), as obtained by polymerisation, polycondensation or polyaddition, for example a polyolefin, such as polyethylene, polypropylene or polyisobutylene, a substituted polyolefin, such as a polymerisation product of vinyl chloride, vinyl acetate, styrene, acrylonitrile, an acrylic acid and/or methacrylic acid ester or butadiene, as well as a copolymer of the mentioned monomers, especially ABS or EVA.

From the series of the polyaddition resins and polycondensation resins there may be mentioned the condensation products of formaldehyde with phenols, so-called phenoplasts, and the condensation products of formaldehyde with urea, thiourea and melamine, so-called aminoplasts, the polyesters used as surface-coating resins, either saturated, such as alkyd resins, or unsaturated, such as maleic resins, and also linear polyesters and polyamides or silicones.

The mentioned high molecular weight compounds may be in the form of single compounds or mixtures, in the form of plastic masses or melts, which may optionally be spun to form fibres.

They may also be in the form of their monomers or in the polymerised state in dissolved form as film formers or binders for surface coatings or printing inks, such as boiled linseed oil, nitrocellulose, alkyd resins, melamine resins, urea-formaldehyde resins or acrylic resins.

The pigmenting of the high molecular weight organic substances with the monoazo compounds of formula (1) according to the invention is carried out, for example, by admixing such a monoazo compound, optionally in the form of a masterbatch, with the substrates using roll mills or mixing or grinding apparatus. The pigmented material is then generally brought into the desired final form by methods known per se, such as calendering, compression moulding, extrusion, coating, casting or by injection moulding. In order to produce non-rigid mouldings or to reduce their brittleness it is often desirable to incorporate so-called plasticisers into the high molecular weight compounds prior to shaping. As plasticisers there may be used, for example, esters of phosphoric acid, phthalic acid or sebacic acid. In the process according to the invention, the plasticisers may be incorporated into the polymers before or after the incorporation of the monoazo compound of formula (1) according to the invention. It is also possible, in order to achieve different shades, to add to the high molecular weight organic materials, in addition to the pigment compositions, also fillers or other colour-imparting constituents, such as white, coloured or black pigments as well as effect pigments, in each case in the desired amount.

For the pigmenting of surface coatings and printing inks, the high molecular weight organic materials and the monoazo compounds of formula (1) according to the invention are finely dispersed or dissolved, optionally together with additives such as fillers, other pigments, siccatives or plasticisers, generally in an organic and/or aqueous solvent or solvent mixture. It is possible to use a procedure in which the individual components are dispersed or dissolved separately or in which a plurality thereof are dispersed or dissolved together and only then all of the components combined.

Accordingly, a further embodiment relates to mass-coloured high molecular weight organic material, comprising
(a) from 0.05 to 70% by weight, based on the sum of (a) and (b), of a monoazo compound of formula (1) according to the invention, and
(b) from 99.95 to 30% by weight, based on the sum of (a) and (b), of a high molecular weight organic material.

The material in question may be either a ready-for-use composition or an article formed therefrom, or a masterbatch, for example in the form of granules. The high molecular weight organic material coloured according to the invention may also comprise conventional additives, for example stabilisers.

Accordingly, a further embodiment relates to a method of mass-colouring high molecular weight organic material, which method comprises incorporating into such material a monoazo compound of formula (1) according to the invention, for example by mixing and processing the high molecular weight organic material together with the pigment composition according to the invention, optionally in the form of a masterbatch, in a manner known per se.

The Examples which follow serve to illustrate the invention. Unless otherwise indicated, parts are parts by weight and percentages are percent by weight. Temperatures are given in degrees Celsius. The relationship between parts by weight and parts by volume is the same as that between grams and cubic centimetres.

### EXAMPLE 1

Preparation of the diazonium: 8.9 g (40 mmol, 98%) of 2-amino4-methyl-5-chlorobenzenesulfonic acid are stirred in 100 ml of deionised water, and 10 ml of aqueous 37% HCI are added thereto. The fine suspension is then cooled to a temperature of 0-5° C. by means of an ice bath, and 15 ml of a 4N sodium nitrite solution are added dropwise thereto. Then, after 2 hours, 0.5 ml of 1N sulfamic acid is added.

Coupling: 15.3 g (46 mmol) of 4-acetoacetylaminonaphtholsulfonic acid sodium salt are dissolved in 200 ml of deionised water. The solution is cooled to a temperature of 0-5° C. and the above diazonium suspension is added dropwise thereto, the pH being maintained at 4.5-5.0 by means of a 30% sodium hydroxide solution. When the addition is complete, the reaction mixture is stirred at room temperature for 30 minutes. The suspension is then heated to a temperature of 80° C. and stirred for 1.5 hours. The resulting yellow suspension is filtered by means of hard filters, washed with 200 ml of deionised water and dried in vacuo at 90° C. 7.6 g of the sodium salt of the compound of formula 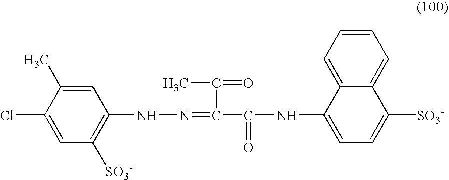
are obtained. 22.5 g of the compound of formula (100) obtained above are stirred in 100 ml of deionised water and heated to a temperature of 80° C. A solution of 2.5 g of calcium chloride (Fluka purum) in 30 ml of deionised water is added. The mixture is diluted with 50 ml of water and adjusted to pH 8.0 with a small amount of NaOH. The mixture is then stirred at that temperature for 2 hours. The resulting yellow suspension is filtered, while hot, through hard filters, washed with 200 ml of deionised water and dried in vacuo at 90° C. 9.7 g of the Ca²⁺/Na⁺ mixed salt of the compound of formula (100), which contains a little water, are obtained, which colours PVC plastics in greenish-yellow shades.

### EXAMPLE 2

Preparation of the diazonium: 4.5 g (20 mmol, 98%) of 2-amino-4-methyl-5-chloro-benzenesulfonic acid are stirred in 100 ml of deionised water, and 7 ml of aqueous 37% HCI are added thereto. The resulting fine suspension is cooled to a temperature of 0-5° C. by means of an ice bath, 5 ml of a 4N sodium nitrite solution are added dropwise and stirring is then carried out for 2 hours.

Coupling: 6.6 g (20 mmol) of 4-acetoacetylaminonaphtholsulfonic acid sodium salt are dissolved in 60 ml of deionised water, and 3.0 g of calcium chloride (Fluka purum) are added. The mixture is then cooled to a temperature of 0-5° C. The above diazonium suspension is added dropwise thereto, the pH being maintained at 5 by means of a 30% sodium hydroxide solution. To suppress foam formation, 20 ml of water and 10 ml of ethanol are added. When the addition of the diazonium is complete, the reaction mixture is stirred first at a temperature of 0-5° C. for 2 hours and then at room temperature for 12 hours. The mixture is then heated to a temperature of 80° C. and maintained at pH 8 with a 30% sodium hydroxide solution. After subsequently being stirred for 2 hours, the yellow suspension is filtered, while hot, through hard filters, washed with 200 ml of deionised water and dried in vacuo at 100C. 9.7 g of the Ca²⁺/Na⁺ mixed salt of the compound of formula (100) are obtained, which colours PVC plastics in greenish-yellow shades.

### EXAMPLE 3

Preparation of the diazonium: 4.5 g (20 mmol, 98%) of 2-amino-4-methyl-5-chloro-benzenesulfonic acid are stirred in 100 ml of deionised water, and 7 ml of aqueous 37% HCI are added thereto. The fine suspension is then cooled to a temperature of 0-5° C. by means of an ice bath, and 5 ml of a 4N sodium nitrite solution are added dropwise thereto. Then, after 2 hours, 0.5 ml of 1N sulfamic acid is added.

Coupling: 6.6 g (20 mmol) of 4-acetoacetylaminonaphtholsulfonic acid sodium salt are dissolved in 100 ml of deionised water, and 2 ml of a 30% sodium hydroxide solution are added. The resulting solution is then added dropwise to the above diazonium suspension, the pH being maintained at 4.5-5.0 by means of a 30% sodium hydroxide solution. The resulting thick suspension is diluted with 100 ml of deionised water and is stirred at a temperature of 0-5° C. for 1.5 hours and then at room temperature for 12 hours. The suspension is then heated to a temperature of 80° C., maintained at pH 8 by addition of a 30% sodium hydroxide solution, and a solution of 2.5 g of calcium chloride (Fluka purum) in 10 ml of deionised water is added. The mixture is then stirred at that temperature for 2 hours. The yellow suspension is filtered, while hot, through hard filters, washed with 250 ml of deionised water and dried in vacuo at 90° C. 10.1 g of the Ca²⁺/Na⁺ mixed salt of the compound of formula (100) are obtained, which colours PVC plastics in yellow shades.

### EXAMPLE 4

Preparation of the diazonium: 9.0 g (40 mmol, 98.5%) of 2-amino-4-chloro-5-methyl-benzenesulfonic acid are stirred in 180 ml of deionised water, and 14 ml of aqueous 37% HCI are added thereto. The fine suspension is then cooled to a temperature of 0-5° C. by means of an ice bath, and 10 ml of a 4N sodium nitrite solution are added dropwise thereto. After 2 hours, 1.0 ml of 1N sulfamic acid is added.

Coupling: 13.2 g (40 mmol) of 4-acetoacetylaminonaphtholsulfonic acid sodium salt are dissolved in 200 ml of deionised water. The solution is cooled to a temperature of 0-5° C. and the above diazonium suspension is added dropwise thereto, the pH being maintained at 4.5-5.0 by means of a 30% sodium hydroxide solution. Any foam formation is suppressed with 5 ml of ethanol and 5 drops of Surfynol 104E. When the addition is complete, the reaction mixture is stirred at 0-5° C. for 2 hours and then at room temperature for 12 hours. The reaction mixture is divided into two samples, each of about 360 ml. The resulting yellow suspension of the first sample is filtered by means of hard filters, washed with 100 ml of water and dried in a vacuum cabinet at 90° C. 10.3 g of the sodium salt of the compound of formula 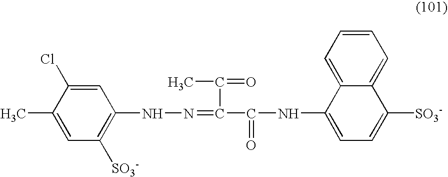
are obtained.

The second portion of the suspension is heated to 80° C. A solution of 3.5 g of calcium chloride (32 mmol) in 10 ml of deionised water is added thereto and the pH is adjusted to 8.0 with a small amount of sodium hydroxide solution. The mixture is then stirred at 80° C. for 2 hours. The resulting yellow suspension is filtered, while hot, through hard filters, washed with 200 ml of deionised water and dried in a vacuum cabinet at,90° C. 10.5 g of the calcium salt of the compound of formula (101) are obtained, which colours PVC plastics in greenish-yellow shades.

### EXAMPLE 5

Preparation of the diazonium: 4.5 g (20 mmol, 98.5%) of 2-amino-4-chloro-5-methyl-benzenesulfonic acid are stirred in 80 ml of deionised water, and 7 ml of aqueous 37% HCI are added thereto. The fine suspension is then cooled to a temperature of 0-5° C. by means of an ice bath, and 5 ml of a 4N sodium nitrite solution are added dropwise thereto. Then, after 2 hours, 1.0 ml of 1N sulfamic acid is added.

Coupling: 6.6 g (20 mmol) of 4-acetoacetylaminonaphtholsulfonic acid sodium salt are dissolved in 60 ml of deionised water. The solution is cooled to a temperature of 0-5° C. and the above diazonium suspension is added dropwise thereto, the pH being maintained at 4.5-5.0 by means of a 30% sodium hydroxide solution. 30 ml of ethanol and 1 ml of acetic acid are then added thereto. When the addition is complete, the reaction mixture is stirred at 0-5° C. for 2 hours. The mixture is then heated to 80° C., a solution of 3.5 g of calcium chloride (32 mmol) in 10 ml of deionised water is added and the pH is adjusted to 8.0 with (a small amount of) NaOH. The mixture is then stirred for a further 2 hours at 80° C. The resulting yellow suspension is filtered, while hot, through hard filters, washed with 300 ml of cold deionised water and dried in a vacuum cabinet at 90° C. 10 g of the Ca²⁺/Na⁺ mixed salt of the compound of formula (101), which contains a little water, are obtained, which colours PVC plastics in greenish-yellow shades.

### EXAMPLE 6

Preparation of the diazonium: 4.5 g (20 mmol, 98.5%) of 2-amino-4-chloro-5-methyl-benzenesulfonic acid are stirred in 80 ml of deionised water, and 7 ml of aqueous 37% HCI are added thereto. The fine suspension is then cooled to a temperature of 0-5° C. by means of an ice bath, and 5 ml of a 4N sodium nitrite solution are added dropwise thereto. Then, after 2 hours, 1.0 ml of 1N sulfamic acid is added.

Coupling: 6.6 g (20 mmol) of 4-acetoacetylaminonaphtholsulfonic acid sodium salt are dissolved in 60 ml of deionised water, and 1 ml of acetic acid is added. The solution is cooled to a temperature of 0-5° C. and the above diazonium suspension is added dropwise thereto, the pH being maintained at 4.5-5.0 by means of a 30% ammonia solution. To suppress any foam formation, 25 ml of isopropanol are added. When the addition is complete, the reaction mixture is stirred at 0-5° C. for 4 hours. The mixture is then heated to 80° C. and adjusted to pH 8.0 with a 30% ammonia solution. The mixture is then stirred at 80° C. for 2 hours. The resulting yellow suspension is filtered, while hot, through hard filters, washed with 1 liter of cold deionised water and dried in a vacuum cabinet at 90° C. 9.2 g of the NH₄⁺/Na⁺ mixed salt of the compound of formula (101) are obtained, which colours PVC plastics in yellow shades.

### EXAMPLE 7

Preparation of the diazonium: 9.98 g (40 mmol, 97%) of 2-amino-4,5-dichlorobenzenesulfonic acid are stirred in 160 ml of deionised water, and 14 ml of an aqueous 37% HCI solution are added thereto. The fine suspension is then cooled to a temperature of 0-5° C. by means of an ice bath, and 10 ml of a 4N sodium nitrite solution are added dropwise thereto. Then, after one hour, 3 ml of a 1 N sulfamic acid solution are added.

Coupling: 13.17 g (40 mmol) of 4-acetoacetylaminonaphtholsulfonic acid sodium salt are dissolved in 120 ml of deionised water. The solution is cooled to a temperature of 0-5° C. and the above diazonium suspension is added dropwise thereto, the pH being maintained at 4.5-5.0 by means of a 30% sodium hydroxide solution. 10 ml of 2-propanol are then added to the suspension. When the addition is complete, the reaction mixture is stirred at 0-5° C. for 3.5 hours and then at room temperature for 12 hours. The reaction mixture is divided into two samples, each of about 320 ml. The first portion of the suspension is heated to 70° C. and then stirred for 2 hours. The resulting yellow suspension is filtered by means of hard filters, washed with 500 ml of water and dried in a vacuum cabinet at 90° C. 8.7 g of the sodium salt of the compound of formula 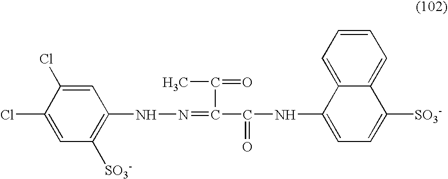
are obtained.

The second portion of the suspension is heated to 70° C. A solution of 3.5 g of calcium chloride (32 mmol) in 10 ml of deionised water is added and the pH is adjusted to 8.0 with a small amount of sodium hydroxide solution. The mixture is then stirred at 70° C. for a further 2 hours. The resulting yellow suspension is filtered, while hot, through hard filters, washed with 200 ml of deionised water and dried in a vacuum cabinet at 90° C. 10.5 g of the Ca²⁺/Na⁺ mixed salt of the compound of formula (102), which contains a little water, are obtained, which colours PVC plastics in greenish-yellow shades.

### EXAMPLE 8

Preparation of the diazonium: 4.5 g (17 mmol, 98.5%) of 2-amino4-methyl-5-chloro-benzenesulfonic acid are stirred in 50 ml of deionised water, and 3.4 g of a 30% potassium hydroxide solution are added. The mixture is heated to 60° C., a solution being formed. The solution is then cooled to 20° C. and, in succession, 4.27 g of an aqueous 37% HCI solution and 4.54 g of a 4N sodium nitrite solution are added dropwise in the course of 15 minutes. After a further 30 minutes, a solution of 2.67 g of calcium chloride in deionised water is added; the mixture is stirred for 5 minutes and then made up to a volume of 110 ml with deionised water.

Coupling: 6.1 g (18 mmol) of 4-acetoacetylaminonaphtholsulfonic acid sodium salt are dissolved in 60 ml of deionised water. A mixture of 1.1 g of acetic acid and 3.2 g of a 30% potassium hydroxide solution in 10 ml of deionised water is added to the coupler solution and then the above diazonium suspension is added within a period of 1 hour and at 20° C., the pH being maintained at about 5.0 by means of a 5% potassium hydroxide solution. The suspension is stirred for a further 10 minutes and then adjusted to pH 9. The reaction mixture is heated to 93° C. within a period of 40 minutes, stirred for a further 10 minutes at that temperature and then cooled to 70° C. The resulting yellow suspension is filtered, while hot, through hard filters, washed with 300 ml of cold deionised water and dried in a vacuum cabinet at 90° C. 10 g of the K⁺/Ca²⁺ mixed salt of the compound of formula (100), which contains a little water, are obtained, which colours PVC plastics in greenish-yellow shades.

1. A monoazo compound of formula 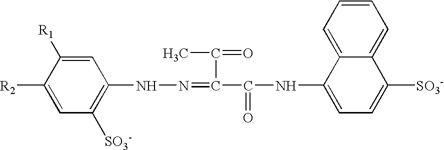
wherein
R₁ is hydrogen, halogen, or C₁-C₄alkyl, and
R₂ is halogen, or C₁-C₄alkyl,
which is present in the form of a salt or a mono-, di-, tri- or tetra-valent mixed salt of different cations.

2. A monoazo compound of formula (1) according to claim 1 wherein R₁ is methyl.

3. (canceled)

4. A monoazo compound of formula (1) according to claim 1 wherein R₁ is chlorine and R₂ is methyl.

5. A monoazo compound of formula (1) according to claim 1 wherein R₁ is methyl and R₂ is chlorine.

6. A Ca²⁺/NH₄⁺ mixed salt of the monoazo compound of formula (1) according to claim 1.

7. A Ca²⁺/Na⁺ mixed salt of the monoazo compound of formula (1) according to claim 1.

8. A Ca²⁺/K⁺ mixed salt of the monoazo compound of formula (1) according to claim 1.

9. A Ca²⁺ salt of the monoazo compound of formula (1) according to claim 1.

10. A process for the preparation of a monoazo compound of formula (1) according to claim 1, which comprises diazotisation of 2-amino-4-methyl-5-chlorobenzenesulfonic acid, 2-amino-4-chloro-5-methylbenzenesulfonic acid or 2-amino-4,5-dichlorobenzenesulfonic acid or a salt or a mixed salt thereof, and coupling to a 4-acetoacetylaminonaphtholsulfonic acid salt.

11. A method of mass-pigmenting high molecular weight organic material, which comprises incorporating into such material a monoazo compound of formula (1) according to claim 1.

12. (canceled)

13. A solid toner, wax transfer ribbon or colour filter containing a monoazo compound according to either 1.

14. A monoazo compound of formula (1) according to claim 1 wherein the cations are selected from Ca²⁺, Na⁺, NH₄⁺, NR₄⁺, H⁺, Li⁺, K⁺, Mg²⁺, Ba²⁺, Sr²⁺, Al³⁺, Pb²⁺, Mn²⁺, Zn²⁺, Cr²⁺, Co²⁺, Fe²⁺, Fe³⁺, Zr⁴⁺ and Cu²⁺ wherein R is C₁-C₆alkyl, in a freely selected ratio.

15. A monoazo compound according to claim 14 wherein R is methyl.
